(19) United States
(12) Patent Application Publication
(10) Pub. No.: US 2021/0369666 A1
(43) Pub. Date: Dec. 2, 2021

(54) DEVICES AND METHODS FOR THE TREATMENT OF A ZOONOTIC HERPESVIRUS INFECTION

(71) Applicants: Thessicar E. ANTOINE, Atlanta, GA (US); Julia HILLARD, Atlanta, GA (US); Rainer ADELUNG, Kiel (DE); Yogendra Kumar MISHRA, Kiel (DE); Deepak SHUKLA, Chicago, IL (US); Mark PRAUSNITZ, Atlanta, GA (US); GEORGIA STATE UNIVERSITY RESEARCH FOUNDATION, INC., Atlanta, GA (US); KIEL UNIVERSITY, Kiel (DE); UNIVERSITY OF ILLINOIS AT CHICAGO, Chicago, IL (US); GEORGIA TECH RESEARCH CORPORATION, Atlanta, GA (US)

(72) Inventors: Thessicar E. ANTOINE, Atlanta, GA (US); Julia HILLARD, Atlanta, GA (US); Rainer ADELUNG, Kiel (DE); Yogendra Kumar MISHRA, Kiel (US); Deepak SHUKLA, Chicago, IL (US); Mark PRAUSNITZ, Atlanta, GA (US)

(21) Appl. No.: 16/345,300

(22) PCT Filed: Oct. 26, 2017
(86) PCT No.: PCT/US17/58482

§ 371 Date: Apr. 26, 2019

Related U.S. Application Data
(60) Provisional application No. 62/412,935, filed on Oct. 26, 2016.

(51) Int. Cl. A61K 31/315; A61K 31/522; A61K 9/00

(52) U.S. Cl. CPC A61K 31/315; A61K 9/0021; A61K 31/522

(57) ABSTRACT

The present disclosure relates to tetrapodal zinc-oxide nanostructures (T-ZONS), microneedle devices, and methods for treating or preventing a zoonotic herpesvirus infection.

### CROSS REFERENCE TO RELATED APPLICATIONS

This application claims the benefit of U.S. Provisional Patent Application Ser. No. 62/412,935 filed Oct. 26, 2016, which is expressly incorporated herein by reference.

### FIELD

The present disclosure relates to tetrapodal zinc-oxide nanostructures (T-ZONS), microneedle devices, and methods for treating or preventing a zoonotic herpesvirus infection.

### BACKGROUND

One of the greatest occupational health risks and hazards that biomedical researchers, animal caretakers, and veterinarians face is the exposure to zoonotic pathogens. The transmission of pathogenic agents to laboratory personnel from vertebrate animals via contact with contaminated animals or materials continues to be an area of high concern despite efforts of commercial vendors to eliminate zoonotic agents from animal colonies. The recognition, diagnosis and treatment of zoonotic diseases in infected laboratory personnel may be delayed based on the presentation of disease. In such cases immediate prophylaxis would significantly control infection spread and dissemination. Zoonosis may be bacterial, viral, or parasitic, or may involve unconventional agents. Zoonotic infections have a global impact on both animal and human health. More than 65% of emerging infectious diseases in humans have been reported to originate from zoonotic pathogens. Among infectious agents associated with the emerging infectious diseases that are zoonotic, viruses are the most likely to pose the greatest threat. The great similarity of genetic, physiological, and behavioral characteristic in human and non-human primates makes the development of effective therapeutics to prevent/control cross-species transmission an area of high interest to investigators worldwide.

Macacine herpesvirus 1 (B-virus) is an alpha-herpesvirus found in macaques that is very closely related to human herpes simplex virus type 1 (HSV-1) and human herpes simplex virus type 2 (HSV-2). B-virus is generally asymptomatic or mild in healthy macaques, however the virus is extremely neurovirulent when transmitted to humans and causes serve and usually fatal encephalitis. While human infections are not common, approximately 80% of untreated cases result in death. As a result of its pathogenic nature, occupational exposure to BV by veterinarians and biomedical researchers who work with macaques is a continuous topic of concern.

With the growing rate of antiviral drug resistance and emerging and re-emerging infections, innovative treatment approaches to fight infections are imperative. Over the past decade various inorganic nanoparticles from inorganic and metal oxides have provided an alternative to traditional therapy, thus achieving unprecedented advantages as enhanced efficiencies, reduced side effects and more targeted localization to sites of disease. Through the collaborative efforts of nanomedicine, uniquely synthesized materials and particles have been functionalized for usage as therapeutic agents, drug delivery systems and diagnostic imaging systems. The recent exploration of the role of zinc oxide (ZnO) to suppress allergen induced inflammation associated with dermatitis, overcome drug resistance via intracellular pH response, and suppress the development and spread of genital lesions associated with HSV-2 infections, have shown promise as new therapeutic agents. However, due to a long list of fatal viruses and the introduction new zoonotic viruses, additional therapeutics are needed for the treatment and prevention of zoonotic infections.

The compositions, devices, and methods disclosed herein address these and other needs.

### SUMMARY

Disclosed herein is a microneedle device for transport of a material across a biological barrier of a subject comprising:

i) a plurality of microneedles each having a base end and a tip;

ii) a substrate to which the base ends of the microneedles are attached or integrated; and

iii) tetrapodal zinc-oxide nanostructures (T-ZONS).

In one aspect, disclosed herein is a method for treating or preventing a zoonotic herpesvirus infection in a subject in need thereof, comprising:

administering to a subject in need thereof a therapeutically effective amount of tetrapodal zinc-oxide nanostructures (T-ZONS).

In another aspect, disclosed herein is a method for treating or preventing a zoonotic herpesvirus infection in a subject in need thereof, comprising:
a) providing a microneedle patch to a subject, wherein the microneedle patch comprises:
   a plurality of microneedles each having a base end and a tip;
   a substrate to which the base ends of the microneedles are attached or integrated; and
   tetrapodal zinc-oxide nanostructures (T-ZONS).
b) inserting the microneedles into a biological barrier, wherein the tetrapodal zinc-oxide nanostructures (T-ZONS) bind to a virus particle in the subject.

In some embodiments, disclosed herein is a method for inhibiting the cell entry, inhibiting the viral replication, or inhibiting the viral spreading of a zoonotic herpesvirus infection comprising:

administering to a subject in need thereof a therapeutically effective amount of tetrapodal zinc-oxide nanostructures (T-ZONS).

In some embodiments, the zoonotic herpesvirus infection is a non-human primate herpesvirus. In some embodiments, the zoonotic herpesvirus infection is a macaque herpesvirus. In some embodiments, the zoonotic herpesvirus infection is macacine herpesvirus 1 (B-virus). In some embodiments, the zoonotic herpesvirus infection is a baboon herpesvirus. In some embodiments, the zoonotic herpesvirus infection is the baboon herpesvirus Papiine herpesvirus 2 (HVP-2).

In some embodiments, the subject is administered the tetrapodal zinc-oxide nanostructures prior to potential herpesvirus exposure. In some embodiments, the subject is administered the tetrapodal zinc-oxide nanostructures subsequent to potential herpesvirus exposure.

In some embodiments, the subject is further administered an additional therapeutic agent. In one embodiment, the additional therapeutic agent is an anti-viral agent. In one embodiment, the additional therapeutic agent is acyclovir.

In one embodiment, the biological barrier can be skin.

### BRIEF DESCRIPTION OF THE DRAWINGS

The accompanying figures, which are incorporated in and constitute a part of this specification, illustrate several aspects described below.

FIG. 1 shows scanning electron microscopy (SEM) investigations on flame grown tetrapodal zinc-oxide nanostructures (T-ZONS) material. (A-C) Series of magnified SEM images (left to right) showing the tetrapodal morphologies of ZnO nano- and micro-structures. The c-axis oriented growth with hexagonal facets can be clearly seen on tetrapod arms (C). (D-E) Energy dispersive X-ray (EDX) elemental map and spectrum confirming the presence of Zn and O elements. The inset in (E) corresponds to the region on which the EDX mapping studies have been performed.

FIG. 2 shows T-ZONS treatment effect on Vero cell viability. (A) Trypan blue dye exclusion assay was used to determine the percentage of live and dead cells following T-ZON treatment. (B) The proliferation of Vero cells 24 hrs post T-ZON treatment as determined by trypan blue. (C) MTS analysis of metabolically activity cells at 24 h post treatment with T-ZONS.

FIG. 3 shows the effect of T-ZONS on Vero cell morphology. (A-D) Vero cells were seeded in a 24 well plate at a concentration of 5×10⁴. Cells were then exposed to T-ZONS for 24 hrs at concentrations of (A) 0 μg/mL, (B) 15.6 μg/mL, (C) 125 μg/mL, or (D) 250 μg/mL and imaged by light microscopy.

FIG. 4 shows T-ZONS treatment significantly reduced HVP-2 infectivity 24-hour post infection. DBG3, GFP reporter cells, were utilized to evaluate the efficacy of T-ZONS to reducing viral entry and replication. Cells were infected with an MOI of 10 and FACS analysis was utilized to determine the percentage of virally infected cells 24 h post infection. FACS data revealed a significant reduction of virus entry. (A-C) The effect of T-ZON treatment on HVP-2 replications was evaluated. DBG3 cells that were protected against entry and replication were GFP negative while those that became infected showed positive GFP signals during FACS analysis (D) The geometric mean intensity of GFP highlights the level of HVP replication that active HSV-1 genes within the reporter cell line (E) Cell count of GFP positive cells were quantified by FITC-A expression

FIG. 5 shows T-ZONS treatment significantly reduces plaque size and plaque number. Vero cells were examined 72 hours post infection to determine the effect of T-ZONS on cell-to-cell spread of HVP-2 virus. The plaque size and shape varied significantly in the presence and absence of T-ZONS. Treatment with T-ZONS caused plaques sizes to be two times smaller than untreated cells. (A) 50 plaques per condition were measured to determine the average diameter of the plaques 72 hours post infection. (B) Plaques were counted to determine T-ZONS ability to inhibit cell-to-cell spread. Treated resulted in significant decreases in plaque number. (C) Histochemical staining of cells with crystal violet allowed plaque morphology to be observed via light microscopy. Images were taken at 100× magnification.

FIG. 6 shows TZONS particles that were synthesized in the presence of ethanol, and/or nitrogen. The newly synthesized particles were smaller is size and dimension and low in toxicity. In panel A, the panel is the naturally synthesized particle. In panel B, the image shows the ethanol-synthesized particles. In panel C, the figure shows the nitrogen synthesized particles.

FIG. 7 shows images of microneedles for the delivery of T-ZONS. In panel A, the image shows plain microneedles filled with casting solution (sugar based media). In panel B, the image shows microneedles loaded with T-ZONS nanoparticles. In panel C, the image shows the actual application step for drug delivery.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

Terms used throughout this application are to be construed with ordinary and typical meaning to those of ordinary skill in the art. However, Applicant desires that the following terms be given the particular definition as defined below.

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

“Activities” of a protein, including those relating to “bioactivity,” include, for example, transcription, translation, intracellular translocation, secretion, phosphorylation by kinases, cleavage by proteases, and/or homophilic and heterophilic binding to other proteins.

The term “administering” refers to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. Administering can be performed using transdermal microneedle-array patches. The term “parenteral” includes subcutaneous, intravenous, intramuscular, intra-articular, intra-synovial, intrasternal, intrathecal, intrahepatic, intralesional, and intracranial injections or infusion techniques.

“Biocompatible” generally refers to a material and any metabolites or degradation products thereof that are generally non-toxic to the recipient and do not cause any significant adverse effects to the subject.

A “composition” is intended to include a combination of active agent and another compound or composition, inert (for example, a detectable agent or label) or active, such as an adjuvant.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.”

As used herein, “conjugated” refers to a non-reversible binding interaction.

As used herein, “displace” refers to interrupting a molecular or chemical interaction between, for example, a protein domain and a peptide, a protein domain and a chemical, a protein domain and a nucleic acid sequence by a chemical, peptide, or nucleic acid having affinity for that specific protein domain than the peptide, chemical, or nucleic acid being displaced.

A “linker” as used herein refers to a molecule that joins adjacent molecules. Generally a linker has no specific biological activity other than to join the adjacent molecules or to preserve some minimum distance or other spatial relationship between them. In some cases, the linker can be selected to influence or stabilize some property of the adjacent molecules, such as the folding, net charge, or hydrophobicity of the molecule.

The terms “peptide,” “protein,” and “polypeptide” are used interchangeably to refer to a natural or synthetic molecule comprising two or more amino acids linked by the carboxyl group of one amino acid to the alpha amino group of another.

The term “carrier” or “pharmaceutically acceptable carrier” means a carrier or excipient that is useful in preparing a pharmaceutical or therapeutic composition that is generally safe and non-toxic, and includes a carrier that is acceptable for veterinary and/or human pharmaceutical or therapeutic use. As used herein, the terms “carrier” or “pharmaceutically acceptable carrier” encompasses can include phosphate buffered saline solution, water, emulsions (such as an oil/water or water/oil emulsion) and/or various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below.

As used herein, the term “polymer” refers to a relatively high molecular weight organic compound, natural or synthetic, whose structure can be represented by a repeated small unit, the monomer (e.g., polyethylene, rubber, cellulose). Synthetic polymers are typically formed by addition or condensation polymerization of monomers. As used herein, the term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of disease), during early onset (e.g., upon initial signs and symptoms of disease), or after an established development of disease symptoms. Prophylactic administration can occur for several days to years prior to the manifestation of symptoms of an infection.

The term “specifically binds,” as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “controlled-release” or “controlled-release drug delivery” or “sustained-release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

### Devices and Methods

Disclosed herein is a microneedle device for transport of a material across a biological barrier of a subject comprising:

i) a plurality of microneedles each having a base end and a tip;

ii) a substrate to which the base ends of the microneedles are attached or integrated; and

iii) tetrapodal zinc-oxide nanostructures (T-ZONS).

In one aspect, disclosed herein is a method for treating or preventing a zoonotic herpesvirus infection in a subject in need thereof, comprising:

administering to a subject in need thereof a therapeutically effective amount of tetrapodal zinc-oxide nanostructures (T-ZONS).

In another aspect, disclosed herein is a method for treating or preventing a zoonotic herpesvirus infection in a subject in need thereof, comprising:
a) providing a microneedle patch to a subject, wherein the microneedle patch comprises:
   a plurality of microneedles each having a base end and a tip;
   a substrate to which the base ends of the microneedles are attached or integrated; and
   tetrapodal zinc-oxide nanostructures (T-ZONS).
b) inserting the microneedles into a biological barrier, wherein the tetrapodal zinc-oxide nanostructures (T-ZONS) bind to a virus particle in the subject.

In some embodiments, disclosed herein is a method for inhibiting the cell entry, inhibiting the viral replication, or inhibiting the viral spreading of a zoonotic herpesvirus infection comprising:

administering to a subject in need thereof a therapeutically effective amount of tetrapodal zinc-oxide nanostructures (T-ZONS).

In some embodiments, the zoonotic herpesvirus infection is a non-human primate herpesvirus. In some embodiments, the zoonotic herpesvirus infection is a macaque herpesvirus. In some embodiments, the zoonotic herpesvirus infection is macacine herpesvirus 1 (B-virus). In some embodiments, the zoonotic herpesvirus infection is a baboon herpesvirus. In some embodiments, the zoonotic herpesvirus infection is the baboon herpesvirus Papiine herpesvirus 2 (HVP-2).

In some embodiments, the subject is administered the tetrapodal zinc-oxide nanostructures prior to potential herpesvirus exposure. In some embodiments, the subject is administered the tetrapodal zinc-oxide nanostructures subsequent to potential herpesvirus exposure.

In some embodiments, the subject is further administered an additional therapeutic agent. In one embodiment, the additional therapeutic agent is an anti-viral agent. In one embodiment, the additional therapeutic agent is acyclovir.

The biological barrier can be can be skin, for example, human skin. The microneedle devices disclosed herein can also be used for controlling transport across tissues other than skin. For example, microneedles can be inserted into the eye across, for example, conjunctiva, sclera, and/or cornea, to facilitate delivery of drugs into the eye. These microneedles may also be inserted into the buccal (oral), nasal, vaginal, or other accessible mucosa to facilitate transport into, out of, or across those tissues. For example, a therapeutic may be delivered across the buccal mucosa for local treatment in the mouth or for systemic uptake and delivery. As another example, microneedle devices may be used internally within the body on, for example, the lining of the gastrointestinal tract to facilitate uptake of orally-ingested drugs or the lining of blood vessels to facilitate penetration of drugs into the vessel wall.

In one embodiment, the microneedle patch is used for the sustained delivery of a therapeutic, for example, T-ZONS alone or in combination with an additional therapeutic.

The microneedles may also comprise a variety of materials, including metals, ceramics, semiconductors, organics, polymers, composites, or a combination thereof. Typical materials of construction include pharmaceutical grade stainless steel, gold, titanium, nickel, iron, tin, chromium, copper, palladium, platinum, alloys of these or other metals, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone), and hyaluronic acid.

The tetrapodal ZnO nanostructures (T-ZONS) can be synthesized by a recently introduced solvent free flame transport synthesis process employing microscopic zinc particles and a sacrificial polyvinyl butyrol (PVB) powder (Mishra Y K, et al. Direct Growth of Freestanding ZnO Tetrapod Networks for Multifunctional Applications in Photocatalysis, UV Photodetection, and Gas Sensing. ACS Appl Mater Interfaces. 2015 Jul. 8; 7(26):14303-16; Mishra Y K, et al. Fabrication of Macroscopically Flexible and Highly Porous 3D Semiconductor Networks from Interpenetrating Nanostructures by a Simple Flame Transport Approach. September 2013, Volume 30, Issue 9, Pages 775-783). The mixture of zinc particles and PVB powder in appropriate weight ratio (1:2) is heated in a muffle furnace at 900° C. for 30 minutes and afterwards the nano- and microscale tetrapodal ZnO structures are formed. Further details about tetrapod growth can be found in previous work (Mishra Y K, et al. Fabrication of Macroscopically Flexible and Highly Porous 3D Semiconductor Networks from Interpenetrating Nanostructures by a Simple Flame Transport Approach. September 2013, Volume 30, Issue 9, Pages 775-783). Ultra Plus Zeiss scanning electron microscopy (SEM) machine which is equipped with energy dispersive X-ray analysis detector (EDX) is utilized for morphological investigations of the grown T-ZONS material. Additional methods for synthesizing tetrapodal ZnO nanostructures (T-ZONS) can be found in U.S. Pat. No. 9,182,399, the entire contents of which are incorporated herein by reference.

In addition to delivery by a microneedle device, the TZONS can also be administered in a medicament for the treatment and/or prophylaxis of conditions caused by zoonotic herpesvirus particles. For example, the medicament can be used topically in the form of suspensions, ointments, creams, and lotions.

In some embodiments, the T-ZONS can be further administered in combination with an additional therapeutic agent. In one embodiment, the additional therapeutic agent is an anti-viral agent. In one embodiment, the anti-viral agent is selected from acyclovir, valacylovir, ganciclovir, an additional nucleoside analogue, or a combination thereof.

In some embodiments, the additional agent to be delivered to the recipient can also be a therapeutic, prophylactic, or diagnostic agent. For example, the agent can be selected from the group consisting of peptides, proteins, carbohydrates, nucleic acid molecules, lipids, organic molecules, biologically active inorganic molecules, and combinations thereof. For example, a wide range of drugs may be formulated for delivery with the present microneedle devices and methods. As used herein, the terms “drug” or “drug formulation” are used broadly to refer to any prophylactic, therapeutic, or diagnostic agent, or other substance that which may be suitable for introduction to biological tissues, including pharmaceutical excipients and substances for tattooing, cosmetics, and the like. The drug can be a substance having biological activity. The drug formulation may include various forms, such as liquid solutions, gels, solid particles (e.g., microparticles, nanoparticles), or combinations thereof. The drug may comprise small molecules, large (i.e., macro-) molecules, or a combination thereof. In representative, not non-limiting, embodiments, the drug can be selected from among immunologic adjuvants (for example, monophosphoryl lipid A (MPLA), aluminum salt (Alum), CpG oliogodeoxynucleotides (ODN)), amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, and viruses. The drug may be selected from suitable proteins, peptides and fragments thereof, which can be naturally occurring, synthesized or recombinantly produced. The drug formulation may further include one or more pharmaceutically acceptable excipients, including pH modifiers, viscosity modifiers, diluents, etc., which are known in the art.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Zinc Oxide Nanostructures: An Efficacious Nanotherapeutic for Blocking Entry of Zoonotic Viruses

Many emerging and re-emerging infectious diseases progress from zoonotic diseases. Despite advances in medicine, a significant occupational risk of contracting these life-threatening diseases exists for veterinarians, biomedical researchers, and animal caregivers, each who first come into contact with animal reservoirs or inadvertent animal hosts. Innovative treatment approaches are imperative. This need coupled with the extensive and growing usage of nanoparticles provided the basis for the studies described here.

Complex shaped tetrapodal zinc oxide nanostructures (T-ZONS) were used in this example to examine their ability to bind to virus and block entry of virions into susceptible cells in culture. It was explored whether Papiine herpesvirus-2 (HVP-2), a nonhuman primate alphaherpesvirus used in lieu of the BSL-4 restricted B virus, could be used to block virion entry in exposed cells. The T-ZONS neutralized virus, inhibiting viral invasion with no toxic effects on infected or uninfected cells.

Specially designed reporter cells, DBG3, which express green fluorescent protein (GFP) under the control of the B virus UL20 promoter, provided a means to evaluate whether virus entry occurred as well as subsequent replication. The use of confocal microscopy provided a means to quantify GFP expression. Finally, viral plaque assays and infectious center assays were performed to quantify virus replication and virus spread. This data revealed for the first time the effective antiviral activity of T-ZONS against zoonotic viruses. The antiviral activity of T-ZONS was mediated by charged surfaces that help in binding with the positively charged residues on the viral envelope. By irreversibly binding virus particles, T-ZONS neutralized virus, reducing viral entry, and thus subsequent replication, and spread. The level of protection observed in these studies shows these specially functionalized nanoparticles can block virus replication at the site of entry.

### Background

Biomedical professionals who work with macaque monkeys or their cells and tissues must be constantly vigilant to minimize exposures to deadly, zoonotic B virus (Macacine herpesvirus 1) (1). Morbidity and mortality due to zoonotic B virus infections, though rare, are high-consequence, life altering events (2). Following potential exposures, immediate first aid is critical, but even when this is successfully performed, virus transmission may have still occurred. The recognition, diagnosis and treatment of zoonotic diseases in infected laboratory personnel may be delayed until apparent disease occurs (3). Any means by which immediate care and prophylaxis can be enhanced have great value for risk reduction.

B virus is an alphaherpesvirus endemic in macaques. Closely related to human herpes simplex virus types 1 and 2 (HSV-1, HSV-2), infection in the natural host is associated with inapparent or mild symptoms in healthy macaques. B virus, however, is neurotropic and neurovirulent when inadvertently transmitted to humans, causing severe, often fatal encephalitis with ascending paralysis in the absence of timely intervention (4, 5). Zoonotic infections are rare, but a sobering fact is that approximately 80% of the time these result in death and/or significant morbidities. Zoonotic infections result from even small scratches as well as bites and splashes. Here, the antiviral efficacy of nanoparticles was investigated to determine whether these can be used immediately on skin or mucosa to bind virus, blocking or reducing virus from entering nerve endings innervating the site of injury.

Over the past decade nanoparticles have provided an alternative to some traditional therapies, enhancing antiviral interventions, reducing side effects, while providing targeted site delivery. Through efforts of research investigators, clinicians can now use uniquely synthesized and functionalized nanostructured materials and nanoparticles as novel interventional tools for drug delivery systems and diagnostic imaging systems (6-8). The successful application of these nanostructures provide testaments to the value of ZnO to suppress allergen-induced inflammation associated with dermatitis (9), overcome drug resistance via intracellular pH responses (10), and reduce development and spread of genital lesions associated with HSV-2 infections (11). ZnO nanoparticles have been widely used in various biomedical engineering applications. However, the recently developed, least cytotoxic branched tetrapodal ZnO structures prepared by the flame approach seem to be very promising for nanomedicine, especially for development of antiviral therapeutics (12-14). Data derived from the use of T-ZONS underscore the promising antiviral capabilities of these to combat HSV-1 and HSV-2 (11, 15, 16). A long list of viruses that can cause fatal infections exists and day-by-day newly emerging viruses appear challenging the capacity of the existing interventions. Therefore, emerging nanoscale materials for use against various such viruses are needed.

To investigate the efficacy of T-ZONS to inhibit entry, replication and spread, Papiine herpesvirus 2 (HVP-2) was used. Papiine herpesvirus 2 (HVP-2) is a nonhuman primate alphaherpesvirus closely related to B virus, endemic in baboons, a close relatives of macaques (17). In addition to biologic similarities, the clinicopathogenesis of HVP-2 in a murine model closely resembles the clinicopathogenesis of B virus in humans and thus HVP-2 provides a non-zoonotic virus model system to predict the efficacy of T-ZONS against B virus entry and subsequent infection.

### Materials and Methods

### T-ZONS: Fabrication and Morphologies

Branched tetrapodal ZnO nanostructures (T-ZONS) were synthesized by a recently introduced solvent-free flame transport synthesis employing zinc microparticles and a sacrificial polymer (polyvinyl butyrol) powder (13, 14). The zinc-polymer mixture in appropriate weight ratio (1:2) was heated in a muffle furnace at 900° C. for 30 minutes to induce growth of branched, nano- and microscale tetrapodal, ZnO spike structures. These and additional details of tetrapod growth were previously described (14).

### Cells and Virus

African green monkey kidney (Vero) cells (ATCC Lot #CCL-81) were purchased from ATCC (Manassas, Va.). Vero cells were passaged in Dulbecco's modified Eagle's medium (DMEM; Sigma Aldrich, St. Louis, Mo.] supplemented with 10% fetal bovine serum (FBS; Atlanta Biological, Atlanta, Ga.), 100 units of penicillin (P), and 100 μg of streptomycin/mL (S) (Sigma Aldrich, St. Louis, Mo.). The DBG3 cells, generously provided by Dr. Richard Eberle, were Vero cells engineered to contain green fluorescent protein (GFP) reporter under the control of the B virus UL20 promoter which is also expressed with replication of HVP-2 (18-20). The DBG3 cells were cultured in DMEM supplemented with 500 μg/mL of Gentamycin for plasmid selection (G-418; ThermoFisher, Grand Island, N.Y.). HVP-2 (Papiine herpesvirus 2, HVP-2), a clinical isolate from the laboratory was propagated and quantified in Vero cells, and stored at −80° C.

### Trypan Blue Exclusion Assay

The DBG3 and Vero cells lines were treated by adding T-ZONS to described growth medium for 24 hours. Viability of treated and untreated cell lines was measured using Trypan blue exclusion assays to determine T-ZONS effect on cell viability post treatment. Briefly, Vero cells were seeded at a density of 5×10⁴ in 24-well, sterile culture plates (Costar, St. Louis, Mo.). The T-ZONS nanowires were suspended in DMEM medium at concentrations of 1000, 500, 150, 100, 50, 25, 10, or 0 μg/mL. At 24, 20 μl of cells were aseptically transferred to sterile 1.5 mL Eppendorf tubes, and incubated at room temperature at 1:1 ratio with 0.4% trypan blue solution (Amresco; Solon, Ohio). Cell viability and cell size were quantified.

### MTS

MTS cytotoxicity assays were also performed 24 hours post treatment. Briefly, Vero cells were seeded at a density of 2×10⁴ in a 96-well culture plate and incubated until 90% confluent. The ZnO nanostructures (1000, 500, 150, 100, 50, 25, 10, or 0 μg/mL) were suspended in complete DMEM medium, and added to the appropriate wells (21). At 24 hours post treatment (hpt), cell cytotoxicities were quantified using a chromogen-based kit (CellTiter Aqueous 96; Promega, Madison, Wis.). Colorimetric detection (A490) was measured using a Powerwave HT microplate spectrophotometer (BioTek, Winooski, Vt.). Data analyses were performed according to kit-manufacturer instructions.

### Viral Plaque Assay

Cell-to-cell transmission was measured by the analysis of plaque formation. A monolayer of Vero cells was seeded in a 24-well plate at density of 5×10⁴ cells per well. Upon reaching ˜90% confluency, cells were treated with 0.1 mg/mL of T-ZONS nanowires and infected at multiplicity of infection (MOI) 0.001 with HVP-2. At 2 hours post-infection (hpi) inocula were removed, cells were washed once with PBS, and 1% methylcellulose was added over each monolayer. Cells were incubated at 37° C. for 72 hours as described above. At the end of the incubation, cells were fixed with methanol for 20 minutes at room temperature and stained with crystal violet. Plaques were counted and assessed at 100× total magnification (Zeiss Axiovert 200).

### Plaque Size Determination

Vero cells were seeded in a 24-well plate at density of 5×10⁴ cells per well. Upon obtaining 90-95% subconfluency, cells were exposed to inoculum mixture containing HVP-2 (MOI 0.001) and T-ZONS or HVP-2 only. At 2 hpi, inocula were removed, cells were washed once with PBS, and 1% methylcellulose added to each monolayer. At 72 hpi plaques were measured at 100× total magnification. The area of each plaque was calculated using Axioversion software (Gottingen, Germany). Measuring the size 30 plaques from each group provided the average area.

### Infectious Spread Assay

The virus spread assay was performed as described previously (21). Briefly, monolayers of DBG3 cells were infected and treated with HVP-2 and T-ZONS, as described above. Following a 2-hour adsorption period with HVP-2, the inoculum was replaced with fresh DMEM culture medium supplemented with (1% FBS, 1% P/S). At 24 hpi the spread of HVP-2 among DBG3 cells was assessed by capturing images of the GFP expressing cell clusters. Fluorescence intensity of images was calculated using ImageJ (22).

### Flow Cytometry

HVP-2 infection-associated induction of GFP expression from DBG3 cells was assayed by flow cytometry, Cells were counted and incubated with HVP-2 (MOI 10). Cells were incubated for 2 hours at 37° C. before inoculum, was removed. The GFP expression, as a result of HVP-2 replication, was quantified using flow cytometry. Approximately 30,000 cells were counted to determine mean fluorescence intensity (MFI). Flow cytometry was performed on a Cyan ADP Analyzer™ (Beckman Coulter, CA) and data were analyzed using FlowJo™ analysis software (FlowJo, LLC, Ashland, Oreg.).

### Statistics

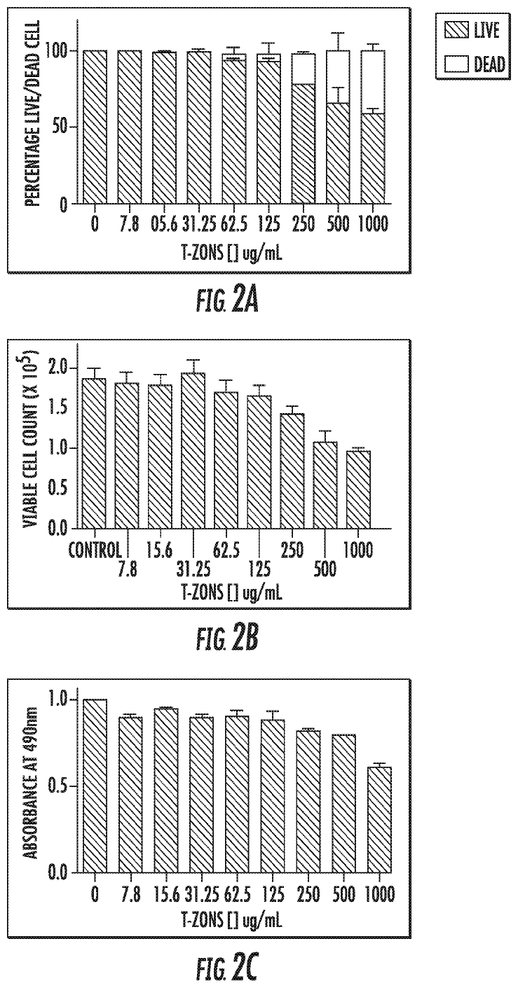

Error bars of all figures represent the S.E.M. of three independent experiments (n=3) unless otherwise specified. Asterisks denote a significant difference as marked in each figure. Data in FIG. 2 was analyzed using two-way ANOVA to compare whether there were significant differences between T-ZON-treated and untreated groups with respect to cell proliferation and/or viability following treatment of each cell line with a dose range of nanoparticles over 24 hrs.

### Results

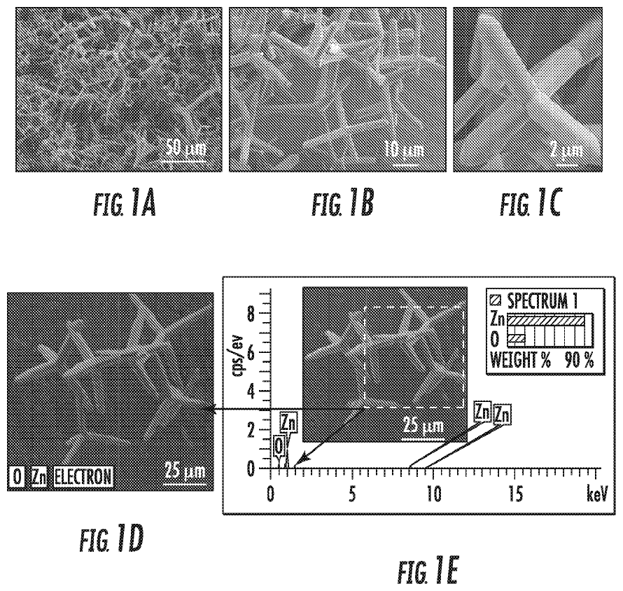

The shapes, composition, sizes, and characteristics of the nanoparticles prepared (T-ZONs) were determined by imaging the using an Ultra Plus Zeiss scanning electron microscope (SEM) equipped with Energy Dispersive X-ray (EDX) analysis detector. FIG. 1 is representative of typical SEM images and EDX elemental maps observed with the ZnO structures used in these studies. Most of the structures exhibited tetrapodal shape with arm diameter in the range of 1-5 μm and lengths in the range of 5-25 μm as shown (panels A-C, low to high) in SEM images in FIG. 1. Corresponding EDX elemental mapping results are shown in panels D-E. The electron beam was focused on a few isolated T-ZONS and corresponding elemental analysis was performed. The EDX elemental map corresponding to inset in FIG. 1E is shown in FIG. 1D confirming the maps of Zn and O elements. No impurities were detected by analyses of images. The chemical compositions of T-ZONS were also confirmed by NMR studies. The weight average and molecular weight distribution were determined to be about 18.8% oxygen and about 81.2% zinc.

### T-ZONS Effect on Cell Viability and Cellular Morphology

A primary concern when evaluating the efficacy of prophylactic application of T-ZONS powder is safety/toxicity to cell cultures used in these studies. Cellular viability and cell morphology were examined to ensure there were no measurable or apparent negative or positive effects of the functionalized nanowires on the cells. Cell viability was determined by trypan blue exclusion assay. Following 24 h treatment with T-ZONS as previously described, cells were detached from the surface of wells by trypsin and treated with the vital stain (Trypan Blue), which is readily taken up by dying cells indicating decreased membrane integrity in negatively impacted cells. FIG. 2A shows an increased in the presence of trypan blue uptake in cells treated at higher concentration of T-ZONS. FIG. 2B revealed a significant reduction in cell number in groups treated with high concentrations of T-ZONS.

The ability of living cells to breakdown tetrazolium after 24 of exposure to T-ZONS was analyzed by MTS assay. FIG. 2C shows the average OD reading produced by Vero cells at the eight treatment concentrations. With limited sample manipulation, the metabolically active cells are able to break down MTS through the actions of dehydrogenase enzymes. As seen in Trypan Blue Assay, T-ZONS induced noteworthy cytotoxicity at high concentrations.

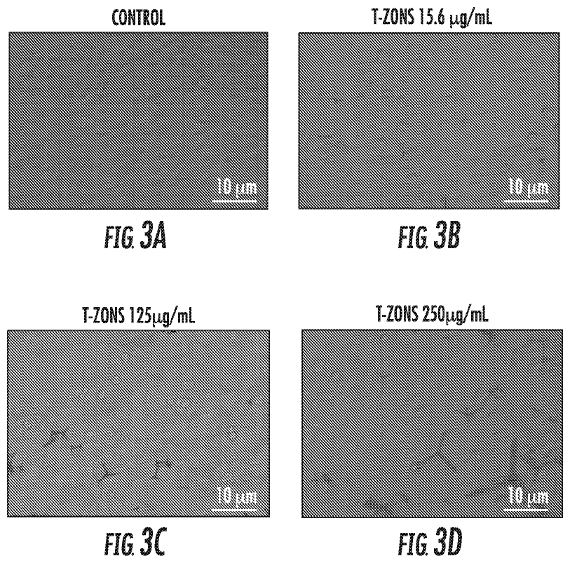

To ensure that T-ZONS treatment did not induce morphological distress, cell images were taken 24 hrs post T-ZON treatment. FIG. 3 shows that T-ZONS treatment had little-to-no effect on Vero cells in terms of cell shape, cell adhesion, and cell diameter

### Infectious Cluster Assay

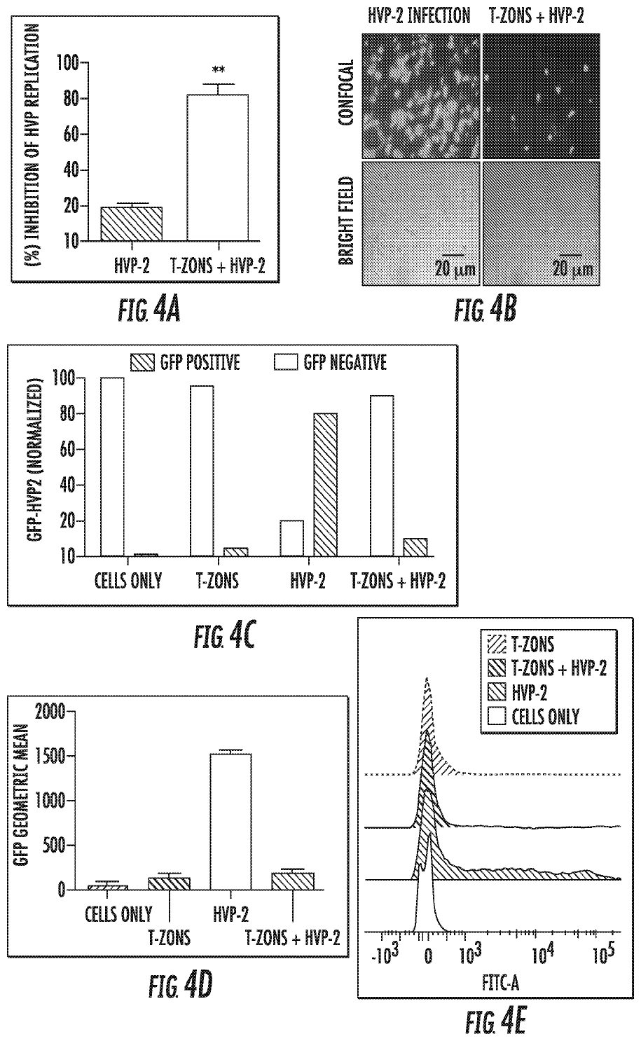

The syncytial nature of alphaherpesviruses is key to spread and immune evasion. To determine the efficacy of T-ZONS in suppressing syncytial formation and cell-to-cell spread of the virus, DBG-3 cells, a specially constructed B virus reporter-containing cell line (19), were used and cell-to-cell spread of virus was captured using confocal microscopy. HVP-2 infected cells were assayed for the presence of infectious clusters. Upon infection with HVP-2 or B virus and/or other related viruses, DBG-3 cells expressed a green-florescent protein allowing the rapid detection of infectious centers. To assess the antiviral properties of T-ZONS, DBG-3 cells in the presence of T-ZONS were infected with HVP-2 (MOI 0.1) for 24 hours following a one-hour adsorption. When analyzing the percentage of GFP expression in cells, it was observed that cells in the absence of T-ZONS expressed a remarkable amount of GFP, which correlated directly with the increased number of virus-infected cells (FIG. 4A). Cells were then imaged (FIG. 4B) to determine the extent to which GFP was expressed within cells. As shown in FIG. 4B, cells treated with T-ZONS displayed a significant reduction in GFP-expressing infectious clusters 24 hour hpi.

### Flow Cytometry

Upon gaining entrance into the cells, HVP-2 initiates replication for the production of new progeny. The usage of DBG-3 reporter cells allows an estimate of the level of replication within each cell by the activation of the GFP-reporter gene. To confirm the microscopic observations of GFP expression following T-ZON treatment, cells were assayed also by flow cytometry. This evaluation allows quantification of GFP expression in DBG-3 resulting from an active virus infection (FIG. 4C). The mean values of GFP expression in FIG. 4D underscored the efficacy of T-ZONS for suppression of viral infection. Cells infected with HVP-2 in the absence of an hour pre-treatment with T-ZONS showed a 3-fold increase in GFP expression relative to treated cells. The histogram in FIG. 4E shows significantly decreased expression of GFP in cells treated with T-ZONS prior to infection when compared to untreated infected cells. This observation revealed the efficacy of T-ZONS to contain and sequester viral replication. Thus, pretreatment of cells prior to virus exposure provides an additional reduction in HVP-2 replication, and thus T-ZONS can provide additional efficacy when used prophylactically.

### Viral Plaque Analysis

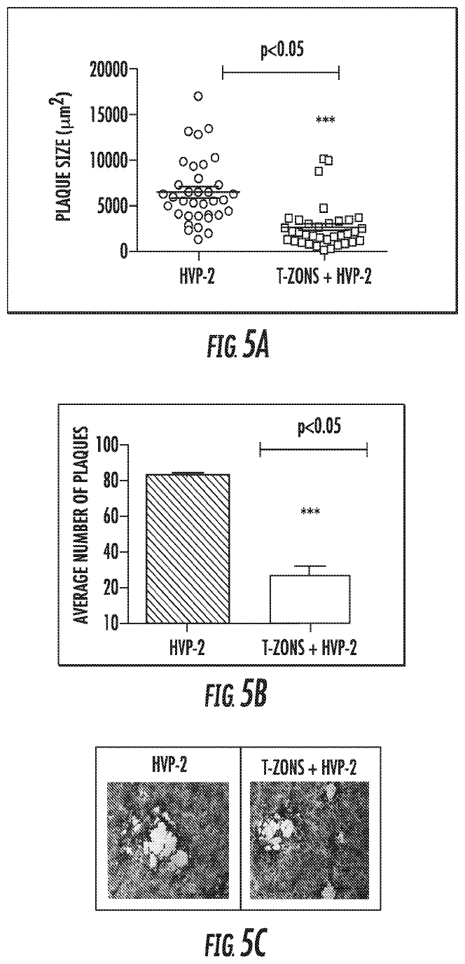

To quantify the effects of T-ZONS on HVP-2 infectivity and replication, a viral plaque assay was performed. By performing plaque analyses, the effect of T-ZONS on HVP-2 growth, replication, transmission and lytic behavior can be assessed. Nearly confluent monolayers of Vero cells were infected with HVP-2 at MOI 0.001 to induce apparent cytopathic effects (CPE) of infection. At two hpi, virus inoculum was removed and cells were overlaid with 1% methylcellulose-containing DMEM to restrict viral diffusion through medium, promoting plaque-formation due to virus replication with lateral spread. At 72 hpi, cells were fixed and stained to quantify the size and numbers of plaques formed following T-ZONS treatment. T-ZONS-treated cells showed fewer plaques than the positive, untreated control, indicating the efficacy of T-ZONS as an antiviral agent, shown in FIG. 5B. As demonstrated in FIG. 5A, the average plaque size of T-ZONS treated cells were also measurably smaller (2628±411.1) when compared to the untreated control (6497±604.8). No apparent effect on the morphology of plaques is seen in FIG. 5C.

### Discussion

With increasing viral threats rising each year and limited numbers of approved antiviral drugs, investigators are challenged to broaden and develop innovative strategies to minimize consequences to human health and wellbeing. Recent exploration of nanoparticle interactions with biological targets has transformed biomedical research and drug development. These interactions have led to nanowire functionalization as a putative microbicidal that traps free-floating virus particles. Here, it was demonstrated for the first time that interactions between functionally modified nanowires and human alphaherpesviruses result in neutralization of infectious particles prior to their entry into cells while also reducing the amount of virus produced by cells that do get infected. Furthermore, these data show the utility of T-ZONS prophylactically to gain added efficacy. The significant role of ZnO in inhibiting viral entry processes highlights the use of nanowires and ZnO as alternatives to viral replication inhibitors, e.g., acyclovir and other nucleoside analogs.

In spite of the ability of current antivirals to effectively treat B-virus exposed patients, administration of these has been unable to completely inhibit virus spread to the peripheral and central nervous systems. Although the B-virus infection is lethal, the investigation of other drugs, as well as drug discovery for therapeutic interventions for zoonotic B virus infections have not been targeted by funding agencies. Zoonotic infections will continue to be a serious and costly consequence for individuals that work with macaques or their cells and tissues. This study provides insight into the usage of T-ZONS particles against HVP-2, and thus B virus, as well as a predictive model for B-virus control during onset of active primary or reactivated infection. The direct targeting ability of T-ZONE-charged surfaces to attract virions away from cell surface receptors is a novel strategy to inhibit B virus infection of targeted skin cells of the epidermal and dermal cells. Additionally, these data indicate that cells show no loss in viability as a result of exposure to the T-ZONS. The assessment of drug safety and efficacy follows the administration of high concentrations.

This study evidences for the first time that concentrated levels of T-ZONS can be easily administered without the fear of compromising cell membrane integrity and viability. The inhibition of virus entry and replication are key functions of any treatment that will be effective against B-virus or similarly acquired infections. The alignment of these results with prior research findings expands the functional role of nanowires to contain infections in humans and non-human primates. Virus spread and dissemination are hallmark characteristics of herpes infections. To properly evaluate the potential of a developing drug or approach to be used against such infections, these processes must be inhibited either by sequestration or interruption of the progression of infection following virus entry. By analyzing cell-to-cell spread and infectious clusters formation, changes in the size of plaques was observed, as well as a reduction of the numbers of plaques. These finding show immediate treatment of zoonotic exposures with T-ZONS can induce a two-pronged effect following entry inhibiting both replication and virus spread.

The ability of T-ZONS to bind virus particles, inhibiting entry and subsequent suppression of virus replication as observed with reduction of virus spread, highlights the key mechanisms of action. Reduction of virus infection ensures limited dissemination from the site of inoculation. The historical usage of ZnO-based treatments against skin irritations rashes and mild infections suggest an allotted timeframe for effect treatment to occur. For example, ZnO-based cream and ointments have been found to be most effective when used during the early onset of disease.

### REFERENCES

1. Yee J L, Vanderford T H, Didier E S, et al. Specific pathogen free macaque colonies: a review of principles and recent advances for viral testing and colony management. J Med Primatol. 2016; 45: 55-78.
2. Epp T, Waldner C. Occupational health hazards in veterinary medicine: zoonoses and other biological hazards. Can Vet J. 2012; 53: 144-50.
3. Haagsma J A, Tariq L, Heederik D J, et al. Infectious disease risks associated with occupational exposure: a systematic review of the literature. Occup Environ Med. 2012; 69: 140-46.
4. Huff J L, Barry P A. B-virus (Cercopithecine herpesvirus 1) infection in humans and macaques: potential for zoonotic disease. Emerg Infect Dis. 2003; 9: 246-50.
5. Keeble S A. B virus infection in monkeys. Ann N Y Acad Sci. 1960; 85: 960-9.
6. Singh R, Lillard J W, Jr. Nanoparticle-based targeted drug delivery. Exp Mol Pathol. 2009; 86: 215-23.
7. Cole L E, Ross R D, Tilley J M, et al. Gold nanoparticles as contrast agents in x-ray imaging and computed tomography. Nanomedicine (Lond). 2015; 10: 321-41.
8. Baetke S C, Lammers T, Kiessling F. Applications of nanoparticles for diagnosis and therapy of cancer. Br J Radiol. 2015; 88: 20150207.
9. Wiegand C, Hipler U C, Boldt S, et al. Skin-protective effects of a zinc oxide-functionalized textile and its relevance for atopic dermatitis. Clin Cosmet Investig Dermatol. 2013; 6: 115-21.
10. Liu J, Ma X, Jin S, et al. Zinc Oxide Nanoparticles as Adjuvant To Facilitate Doxorubicin Intracellular Accumulation and Visualize pH-Responsive Release for Overcoming Drug Resistance. Mol Pharm. 2016; 13: 1723-30.
11. Antoine T E, Hadigal S R, Yakoub A M, et al. Intravaginal Zinc Oxide Tetrapod Nanoparticles as Novel Immunoprotective Agents against Genital Herpes. J Immunol. 2016; 196: 4566-75.
12. Papavlassopoulos H, Mishra Y K, Kaps S, et al. Toxicity of functional nano-micro zinc oxide tetrapods: impact of cell culture conditions, cellular age and material properties. PloS one. 2014; 9: e84983.
13. Mishra Y K, Modi G, Cretu V, et al. Direct Growth of Freestanding ZnO Tetrapod Networks for Multifunctional Applications in Photocatalysis, UV Photodetection, and Gas Sensing. ACS Appl Mater Interfaces. 2015; 7: 14303-16.
14. Mishra Y K, Kaps S, Schuchardt A, et al. Fabrication of Macroscopically Flexible and Highly Porous 3D Semiconductor Networks from Interpenetrating Nanostructures by a Simple Flame Transport Approach. Part Part Syst Char. 2013; 30: 775-83.
15. Mishra Y K, Adelung R, Rohl C, et al. Virostatic potential of micro-nano filopodia-like ZnO structures against herpes simplex virus-1. Antiviral research. 2011; 92: 305-12.
16. Antoine T E, Mishra Y K, Trigilio J, et al. Prophylactic, therapeutic and neutralizing effects of zinc oxide tetrapod structures against herpes simplex virus type-2 infection. Antiviral research. 2012; 96: 363-75.
17. Rogers K M, Ritchey J W, Payton M, et al. Neuropathogenesis of herpesvirus papio 2 in mice parallels infection with Cercopithecine herpesvirus 1 (B virus) in humans. J Gen Virol. 2006; 87: 267-76.
18. Rogers K M, Ealey K A, Ritchey J W, et al. Pathogenicity of different baboon herpesvirus papio 2 isolates is characterized by either extreme neurovirulence or complete apathogenicity. Journal of virology. 2003; 77: 10731-9.
19. Black D H, Saliki J T, Eberle R. Development of a green fluorescent protein reporter cell line to reduce biohazards associated with detection of infectious Cercopithecine herpesvirus 1 (monkey B virus) in clinical specimens. Comparative medicine. 2002; 52: 534-42.
20. Wolf R F, Rogers K M, Blewett E L, et al. A naturally occurring fatal case of Herpesvirus papio 2 pneumonia in an infant baboon (Papio hamadryas anubis). Journal of the American Association for Laboratory Animal Science: JAALAS. 2006; 45: 64-8.
21. Trigilio J, Antoine T E, Paulowicz I, et al. Tin oxide nanowires suppress herpes simplex virus-1 entry and cell-to-cell membrane fusion. PloS one. 2012; 7: e48147.
22. Schneider C A, Rasband W S, Eliceiri K W. NIH Image to ImageJ: 25 years of image analysis. Nat Methods. 2012; 9: 671-5.

### Example 2. Alternative Synthesis of T-ZONS

T-ZONS were previously used in the treatment of human herpes simplex type 1 and type-2 infections. This unique particle contains virus trapping ability to the simplex capsid thus neutralizing virus in suspension mixtures and preventing them to enter and infect cells (as described in Antoine et al. Intravaginal Zinc Oxide Tetrapod Nanoparticles as Novel Immunoprotective Agents against Genital Herpes, J. Immunology, 2016 Jun. 1; 196(11):4566-75). PVB synthesized particles, though effective in suppression of virus infection size along the micro scale compared to the nano-scale hindered its usage in drug delivery methods. To address this problem, particles were synthesized in the presence of ethanol, and/or nitrogen. The newly synthesized particles were smaller is size and dimension and low in toxicity. FIG. 6 shows the effect of ethanol and nitrogen had on the size of zinc oxide particles. In FIG. 6, the panel on the left is the naturally synthesized particle. The middle image shows the ethanol-synthesized particles, and the right figure shows the nitrogen synthesized particles.

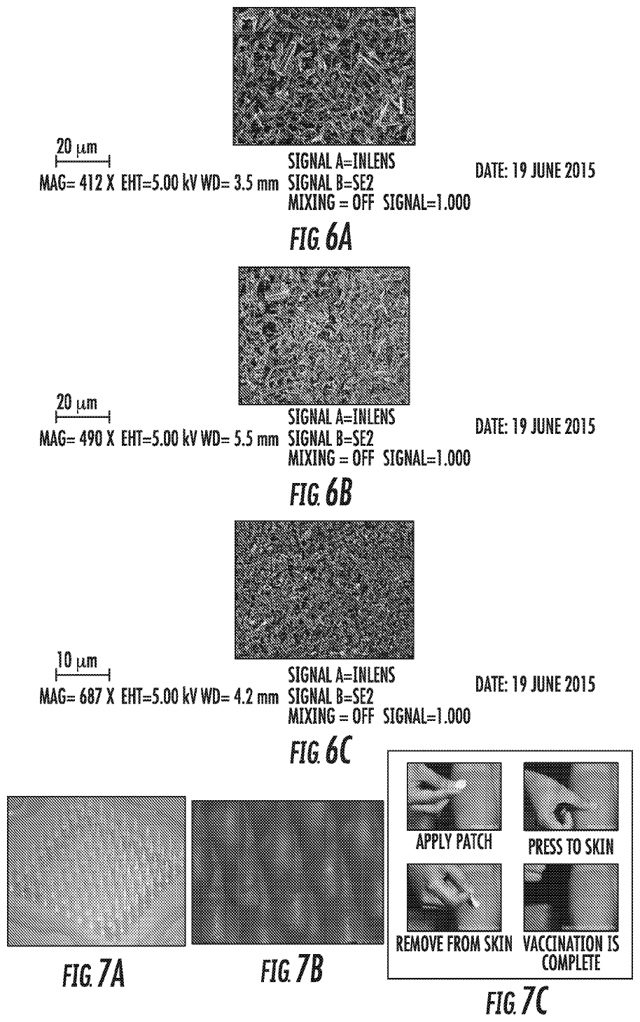

Smaller particle size can improve usage in microneedles at microbicidal concentrations. The ability to cast particles within solution that can be administered by transdermal delivery, expands the application of particles from topical use. The capitation of particles into microneedles allows the non-invasive introduction of particles into a putative site of infection. In FIG. 7, the left image shows plain microneedles filled with casting solution (sugar based media). The image in the middle shows microneedles loaded with T-ZONS nanoparticles, and the image to the right shows the actual application step for drug delivery.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A microneedle device for transport of a material across a biological barrier of a subject comprising:
a plurality of microneedles each having a base end and a tip;
a substrate to which the base ends of the microneedles are attached or integrated; and
tetrapodal zinc-oxide nanostructures (T-ZONS).

2. The device of claim 1, wherein the tetrapodal zinc-oxide nanostructures (T-ZONS) are loaded into the tip of the microneedles.

3. The device of claim 1, wherein the biological barrier is skin.

4. The device of claim 1, wherein the subject is a human.

5. The device of claim 1, wherein the subject is a non-human primate.

6. The device of claim 5, wherein the subject is a macaque.

7. The device of claim 1, comprising an additional agent.

8. The device of claim 7, wherein the additional agent is an anti-viral agent.

9. The device of claim 7, wherein the additional agent is an acyclovir.

10. A method for treating or preventing a zoonotic herpesvirus infection in a subject in need thereof, comprising:
providing a microneedle patch to a subject, wherein the microneedle patch comprises:
a plurality of microneedles each having a base end and a tip;
a substrate to which the base ends of the microneedles are attached or integrated; and
tetrapodal zinc-oxide nanostructures (T-ZONS);
inserting the microneedles into a biological barrier, wherein the tetrapodal zinc-oxide nanostructures (T-ZONS) bind to a virus particle in the subject.

11. The method of claim 10, wherein the zoonotic herpesvirus infection is a herpesvirus.

12. The method of claim 10, wherein the zoonotic herpesvirus infection is a non-human primate herpesvirus.

13. The method of claim 10, wherein the zoonotic herpesvirus infection is a macaque herpesvirus.

14. The method of claim 10, wherein the zoonotic herpesvirus infection is macacine herpesvirus 1 (B-virus).

15. The method of claim 10, wherein the zoonotic herpesvirus infection is a baboon herpesvirus.

16. The method of claim 10, wherein the zoonotic herpesvirus infection is baboon herpesvirus Papiine herpesvirus 2 (HVP-2).

17. The method of claim 10, wherein the tetrapodal zinc-oxide nanostructures are administered prior to potential herpesvirus exposure.

18. The method of claim 10, wherein the tetrapodal zinc-oxide nanostructures are administered subsequent to potential herpesvirus exposure.

19. The method of claim 10, wherein the subject is administered an additional agent.

20.-25. (canceled)

26. A method for treating or preventing a zoonotic herpesvirus infection in a subject in need thereof, comprising administering to the subject a therapeutically effective amount of tetrapodal zinc-oxide nanostructures (T-ZONS).

27.-41. (canceled)
